(19) United States
(12) Patent Application Publication
(10) Pub. No.: US 2020/0318168 A1
(43) Pub. Date: Oct. 8, 2020

(54) UPCONVERSION NANOPARTICLE-BASED MOLECULAR PROBES AND METHODS OF USE

(71) Applicant: The Board of Regents of the University of Oklahoma, Norman, OK (US)

(72) Inventors: Chuanbin Mao, Norman, OK (US); Yueyi Sun, Norman, OK (US)

(21) Appl. No.: 16/838,403

(22) Filed: Apr. 2, 2020

Related U.S. Application Data
(60) Provisional application No. 62/829,727, filed on Apr. 5, 2019.

(51) Int. Cl. C12Q 1/6818; C12Q 1/6876

(52) U.S. Cl. CPC C12Q 1/6818; C12Q 2600/178; C12Q 1/6876

(57) ABSTRACT

Oligonucleotide probes and kits containing same are disclosed, along with methods of use thereof. The oligonucleotide probe comprises a nucleic acid probe sequence comprising a hairpin loop and having a reporter moiety linked to a first end thereof and a quencher moiety linked to a second end thereof. In certain embodiments, the reporter moiety is an upconversion nanoparticle, and the quencher moiety is a gold nanoparticle. The quencher moiety quenches the reporter moiety when the nucleic acid probe sequence is not bound to a complementary target nucleic acid sequence, and the reporter moiety becomes unquenched when the nucleic acid probe sequence binds to a complementary target nucleic acid sequence.

### CROSS REFERENCE TO RELATED APPLICATIONS/INCORPORATION BY REFERENCE STATEMENT

The present application claims priority under 35 U.S.C. § 119(e) to U.S. Ser. No. 62/829,727, filed Apr. 5, 2019. The entirety of the above-referenced patents and patent applications are hereby expressly incorporated by reference herein.

### STATEMENT REGARDING FEDERALLY SPONSORED RESEARCH AND DEVELOPMENT

Not Applicable.

### BACKGROUND

Molecular beacons (MBs) are DNA-based oligonucleotide probes that are designed for the detection of DNA or RNA fragments. A DNA hairpin structure is used to position quencher and fluorophore in close proximity. Only when a target-recognizing sequence of the probe has bound with a complementary target sequence does the beacon emit a fluorescent signal for detection. MBs are useful, for example, for disease monitoring and the study of protein-DNA interactions, etc. Currently, MBs mainly use fluorescent molecules as fluorophores. However, the fluorescent molecules are expensive, have a relatively high background, and are susceptible to photobleaching. It is to mitigating these drawbacks that the embodiments of the present disclosure are directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments of the present disclosure are hereby illustrated in the appended drawings. It is to be noted, however, that the appended drawings only illustrate several typical embodiments and are therefore not intended to be considered limiting of the scope of the inventive concepts disclosed herein. The figures are not necessarily to scale, and certain features and certain views of the figures may be shown as exaggerated in scale or in schematic in the interest of clarity and conciseness.

FIG. 1A is a schematic diagram demonstrating MB synthesis in accordance with the present disclosure.

FIG. 1B is a schematic showing how the MB of FIG. 1A interacts with a target RNA to form a complex that when irradiated with a wavelength emits an emission wavelength.

FIG. 2 shows a schematic diagram of a synthesis method for an alternate MB synthesis method for multiple-UCNP MB.

FIG. 3 shows a transmission electron microscope (TEM) image (left) and an emission spectrum (right) of up-conversion nanoparticles (UCNPs). The average diameter of the UCNPs is 30 nm. The scale bar indicates 50 nm. The excitation wavelength of UCNP is 980 nm; the emission peak is at 541 nm.

FIG. 4 shows a TEM image (left) and an emission spectrum (B-right) of gold nanoparticles (AuNPs). The average diameter of AuNPs is 20 nm. The scale bar indicates 50 nm. The absorption peak of AuNP is at 546 nm.

FIG. 5 shows TEM images (a, b) of NP-based MB. The red arrows point to AuNPs. The scale bar indicates 50 nm.

FIG. 6 shows an emission spectrum of UCNPs (blue) and NP-based MBs (orange). Same concentrations of UCNPs are detected for spectrum in UCNP solution and NP-based MB solution; the fluorescence of MB is significantly lower than UCNP, and the peak does not shift.

FIG. 7 shows a Peak fit image of an UCNP emission spectrum. The emission spectrum of UCNP is fitted as 4 peaks by Origin® 8.6 graphing software (OriginLab Corporation, Northampton, Mass.).

FIG. 8 shows emission intensity of UCNPs versus various target DNA concentrations in aqueous solution. Intensity of peaks is directly correlated with concentration of target DNA in the aqueous solution.

FIG. 9 shows emission intensity of UCNPs versus various target RNA concentrations in aqueous solution. Intensity of peaks is directly correlated with concentration of target RNA in the aqueous solution.

FIG. 10 shows emission intensity of UCNPs versus target RNA concentration in plasma solution. Intensity of peaks is directly correlated with concentration of target RNA in the plasma solution.

### DETAILED DESCRIPTION

The present disclosure, in at least certain non-limiting embodiments, is directed to small DNA- or RNA-detecting molecular probes, also referred to herein as molecular beacons (MBs), that utilize upconversion nanoparticles (UCNPs) as reporter groups in place of fluorophores and thereby provide MBs having ultra-low background to background-free properties. This is due to the UCNP having an excitation wavelength which is longer than the emitting wavelength, i.e., the emission light of the UCNP has a higher energy than the excitation light. The MBs further include a quencher moiety, which may be any suitable quenching particle that functions to quench the emission from the UCNPs until the probe sequence has bound to a target nucleic acid sequence; in at least certain non-limiting embodiments, gold nanoparticles (AuNPs) are used as a quencher moiety.

Before further describing various embodiments of the compositions, kits, and methods of the present disclosure in more detail by way of exemplary description, examples, and results, it is to be understood that the embodiments of the present disclosure are not limited in application to the details of the compositions, kits, and methods as set forth in the following description. The embodiments of the compositions, kits, and methods of the present disclosure are capable of being practiced or carried out in various ways not explicitly described herein. As such, the language used herein is intended to be given the broadest possible scope and meaning, and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. All of the compositions, kits, and methods of production and application and use thereof disclosed herein can be made and executed without undue experimentation in light of the present disclosure. While the compositions, kits, and methods of the present disclosure have been described in terms of particular embodiments, it will be apparent to those of skill in the art that variations may be applied to the compositions, kits, and/or methods and in the steps or in the sequence of steps of the methods described herein without departing from the concept, spirit, and scope of the inventive concepts as described herein. All such similar substitutions and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein. In particular, the entirety of the following patents and published patent application are specifically and individually incorporated herein by reference: U.S. Pat. Nos. 10,246,749; 10,179,177; 10,131,905; 10,093,967; 10,047,388; 9,956,426; 9,995,741; 9,926,603; 9,719,088; 9,562,232; 9,556,379; and 9,410,956; and U.S. Patent Application Publication No. US 2019/0076526.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the objects, or study subjects. As used herein, the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Further, all references to one or more embodiments or examples are to be construed as non-limiting to the claims.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 300 nm to 2500 nm therefore refers to and includes all values or ranges of values, and fractions of the values and integers within said range, including for example, but not limited to, 400 nm to 2250 nm, 400 nm to 2000 nm, 600 nm to 2250 nm, 600 nm to 2000 nm, 400 nm to 1750 nm, 750 nm to 2000 nm, 750 nm to 1750 nm, 750 nm to 1600 nm, 400 nm to 1600 nm, and 800 nm to 1200 nm. Any two values within the range of 300 nm to 2500 nm, therefore, can be used to set the lower and upper boundaries of a range in accordance with the embodiments of the present disclosure.

The term “pharmaceutically acceptable” refers to compounds and compositions which are suitable for administration to humans and/or animals without undue adverse side effects such as toxicity, irritation, and/or allergic response commensurate with a reasonable benefit/risk ratio.

By “biologically active” is meant the ability to modify the physiological system of an organism without reference to how the active agent has its physiological effects.

As used herein, “pure,” “substantially pure,” or “isolated” means an object species is the predominant species present (i.e., on a molar basis, it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein, the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein, the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer to a warm-blooded animal, particularly a mammal or bird. Non-limiting examples of animals within the scope and meaning of this term include dogs, cats, rats, mice, guinea pigs, horses, goats, cattle, sheep, zoo animals, Old and New World monkeys, non-human primates, and humans. The term “sample of biological origin” refers to a sample obtained from a source comprising living organisms or a sample from a living organism. Where the living organism is a warm-blooded animal, the sample may be derived, for example, from a solid tissue or organ, or from a liquid tissue such as but not limited to blood, urine, serum, cerebrospinal fluid, sputum, tears, milk, pus, ascites, or interstitial fluid.

“Treatment” refers to therapeutic treatments. “Prevention” refers to prophylactic treatment measures to stop a condition from occurring. The term “treating” refers to administering the composition to a patient for therapeutic purposes, and may result in an amelioration of the condition or disease.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation, and allergic response) commensurate with a reasonable benefit/risk ratio when used in the manner of the present disclosure. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom(s) thereof. A detectable or measurable improvement includes a subjective or objective decrease, reduction, inhibition, suppression, limit, or control in the occurrence, frequency, severity, progression, or duration of the condition, or an improvement in at least one symptom or an underlying cause or a consequence of the condition, or a reversal of the condition. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences, or underlying causes associated with the condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition (e.g., stabilizing), over a short or long duration of time (e.g., seconds, minutes, hours).

As used herein, “micro RNA” or “miRNA” means a small, noncoding RNA sequence of about 10 to about 50 nucleotides, or about 18 to about 25 nucleotides, or about 20 to about 23 nucleotides in length that can be detected in a biological specimen. Examples of miRNAs that can be detected using the compositions and methods of the present disclosure include, but are not limited to, miRNAs described in U.S. Pat. Nos. 10,093,967 (Table 1); U.S. Pat. Nos. 10,246,749; 10,047,388; 10,131,905; 9,926,603; 9,719,088; 9,562,232; and 9,410,956.

As used herein, the term “nanoparticle” includes particles having an average diameter in a range of about 5 nm to about 200 nm, including, but not limited to, particles in a range of about 5 nm to about 100 nm, about 10 nm to about 50 nm, and about 15 nm to about 40 nm.

As used herein, the term “upconversion” (or “upconverting”) refers to a process in which the sequential absorption of two or more photons by a material leads to the emission of light from the material at a shorter wavelength than the excitation wavelength. The term “upconversion nanoparticle” (UCNP) refers to a nanoparticle capable of upconversion when irradiated with a particular excitation wavelength(s). Examples of UCNPs that can be used herein are shown in the examples below. Other examples of UCNPs that can be used in certain non-limiting embodiments of the present disclosure include, but are not limited to, those identified in U.S. Pat. Nos. 9,556,379; 9,956,426; 9,995,741; and 10,179,177, and U.S. Patent Application Publication No. US 2019/0076526.

Certain non-limiting embodiments of the present disclosure are directed to an oligonucleotide probe that comprises: a nucleic acid probe sequence, a reporter moiety linked to a first end of the nucleic acid probe sequence, and a quencher moiety linked to a second end of the nucleic acid probe sequence. The oligonucleotide probe is utilized to detect a complementary target nucleic acid sequence; the quencher moiety quenches the reporter moiety when the nucleic acid probe sequence is not bound to a complementary target nucleic acid sequence, but the reporter moiety becomes unquenched when the nucleic acid probe sequence binds to a complementary target nucleic acid sequence.

The complementary target nucleic acid sequence detected by the oligonucleotide probe may be any target nucleic acid sequence disclosed or otherwise contemplated herein. Non-limiting examples of target nucleic acid sequences include those listed in Table 2.

In certain non-limiting embodiments, the reporter moiety is an upconversion nanoparticle. Any upconversion nanoparticles disclosed herein or otherwise known in the art may be utilized in accordance with the present disclosure, so long as the oligonucleotide probe containing same can function as described herein.

In certain non-limiting embodiments, the quencher moiety is a gold nanoparticle. Any gold nanoparticles disclosed herein or otherwise known in the art may be utilized in accordance with the present disclosure, so long as the oligonucleotide probe containing same can function as described herein.

In a particular (but non-limiting) embodiment, the gold nanoparticle of the quencher moiety comprises apophyses.

The nucleic acid probe sequence of the oligonucleotide probe may be any nucleic acid sequence (including DNA and/or RNA sequences) that allows the oligonucleotide probe to function as described herein. In particular (but non-limiting) embodiments, the nucleic acid probe sequence comprises a DNA sequence that is complementary to an RNA sequence to be probed (i.e., the complementary target nucleic acid sequence is an RNA sequence). In a particular (but non-limiting) embodiment, the RNA is a micro RNA. In other particular (but non-limiting) embodiments, the nucleic acid probe sequence comprises a DNA sequence that is complementary to a DNA sequence to be probed (i.e., the complementary target nucleic acid sequence is a DNA sequence).

In particular (but non-limiting) embodiments, the nucleic acid probe sequence comprises a hairpin loop.

Certain non-limiting embodiments of the present disclosure are directed to a kit containing one or more of any of the oligonucleotide probes described or otherwise contemplated herein. In particular (but non-limiting) embodiments, the kit contains two or more oligonucleotide probes. When two or more oligonucleotide probes are present, the nucleic acid probe sequences of the two or more oligonucleotide probes may differ from one another, and the reporter moieties of the two or more oligonucleotide probes may differ from one another.

The one or more oligonucleotide probes may be present in the kit in any form that allows the kit to perform in accordance with the present disclosure. For example, but not by way of limitation, the oligonucleotide probe(s) may be provided in a single, individual unit/amount/aliquot, or multiple units/amounts/aliquots of the oligonucleotide probe(s) may be provided within the kit.

In addition to the components described in detail herein above, the kits may further contain other component(s)/reagent(s) for performing any of the particular methods described or otherwise contemplated herein. The nature of these additional component(s)/reagent(s) will depend upon the particular diagnostic/screening assay format and/or the particular test sample to be screened, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

In addition, the kit can further include a set of written instructions explaining how to use one or more components of the kit. A kit of this nature can be used in any of the methods described or otherwise contemplated herein.

Certain non-limiting embodiments of the present disclosure are directed to a method of screening a sample for a target nucleic acid sequence (wherein the target nucleic acid sequence can be any of the target nucleic acid sequences disclosed or otherwise contemplated herein). In the method, any of the oligonucleotide probes disclosed or otherwise contemplated herein is obtained (wherein the nucleic acid probe sequence of the oligonucleotide probe is complementary to the target nucleic acid sequence), and the oligonucleotide probe is combined with the sample, thereby forming an oligonucleotide probe-target nucleic acid complex when the target nucleic acid sequence is present in the sample. In addition, the reporter moiety of the oligonucleotide probe becomes unquenched when the nucleic acid probe sequence binds to the target nucleic acid sequence and forms the oligonucleotide probe-target nucleic acid complex. The sample is then analyzed to detect or measure the oligonucleotide probe-target nucleic acid complex.

Certain non-limiting embodiments of the present disclosure are directed to a method of screening a sample for a target RNA sequence (such as, but not limited to, a micro RNA). In the method, any of the oligonucleotide probes disclosed or otherwise contemplated herein is obtained (wherein the nucleic acid probe sequence of the oligonucleotide probe is complementary to the target RNA sequence), and the oligonucleotide probe is combined with the sample, thereby forming an oligonucleotide probe-target RNA complex when the target RNA sequence is present in the sample. The sample is then analyzed to detect or measure the oligonucleotide probe-target RNA complex.

Certain non-limiting embodiments of the present disclosure are directed to a method of screening a sample for a target DNA sequence. In the method, any of the oligonucleotide probes disclosed or otherwise contemplated herein is obtained (wherein the nucleic acid probe sequence of the oligonucleotide probe is complementary to the target DNA sequence), and the oligonucleotide probe is combined with the sample, thereby forming an oligonucleotide probe-target DNA complex when the target DNA sequence is present in the sample. The sample is then analyzed to detect or measure the oligonucleotide probe-target DNA complex.

Certain non-limiting embodiments of the present disclosure are directed to a method of screening a sample for two or more target nucleic acid sequences (wherein each of the target nucleic acid sequences can be any of the target nucleic acid sequences disclosed or otherwise contemplated herein). The method utilizes two or more of any of the oligonucleotide probes disclosed or otherwise contemplated herein (wherein the nucleic acid probe sequence of each of the oligonucleotide probes is complementary to a corresponding target nucleic acid sequence), wherein the nucleic acid probe sequences and the reporter moieties of each of the oligonucleotide probes differ from the nucleic acid probe sequences and reporter moieties of the other oligonucleotide probe(s). The two or more oligonucleotide probes are combined with the sample, and an oligonucleotide probe-target nucleic acid complex is formed when a target nucleic acid sequence complementary to the nucleic acid probe sequence of one or more oligonucleotide probes is present in the sample. The sample is then analyzed to detect or measure the oligonucleotide probe-target nucleic acid complex for each oligonucleotide probe present.

That is, when two oligonucleotide probes (a first oligonucleotide probe and a second oligonucleotide probe) are utilized in the method, the first and second oligonucleotide probes are combined with the sample; a first oligonucleotide probe-target nucleic acid complex is formed when the first target nucleic acid sequence is present in the sample, and a second oligonucleotide probe-target nucleic acid complex is formed when the second target nucleic acid sequence is present in the sample. When these complexes are formed, the reporter moiety of the oligonucleotide probe present in the complex becomes unquenched when the nucleic acid probe sequence binds to its target nucleic acid sequence. The sample is then analyzed to detect or measure the first oligonucleotide probe-target nucleic acid sequence complex and the second oligonucleotide probe-target nucleic acid sequence complex.

In particular (but non-limiting) embodiments, the sample to be screened in any of the methods described above or otherwise contemplated herein is of biological origin.

The complementary target nucleic acid sequence(s) detected by the methods of the present disclosure may be any target nucleic acid sequence disclosed or otherwise contemplated herein. Non-limiting examples of target nucleic acid sequences include those listed in Table 2.

### EXAMPLES

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures, and methods.

### Example 1

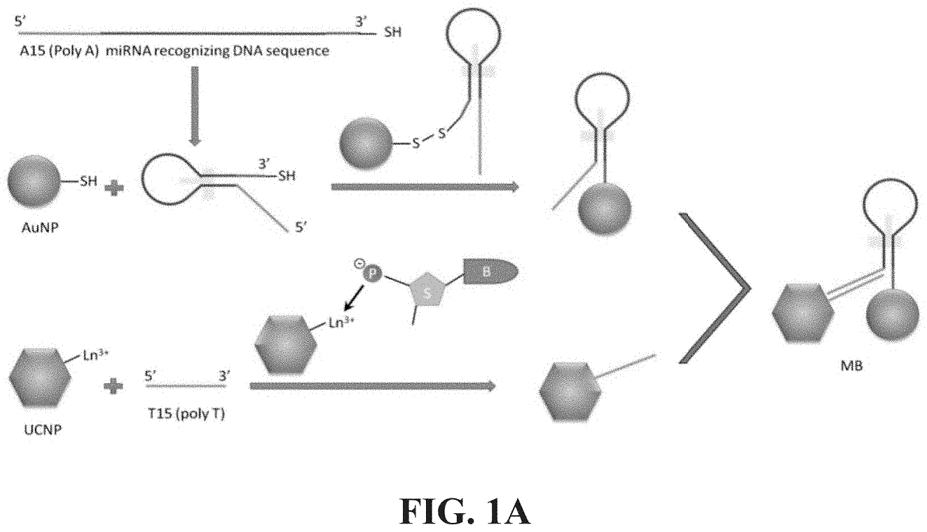

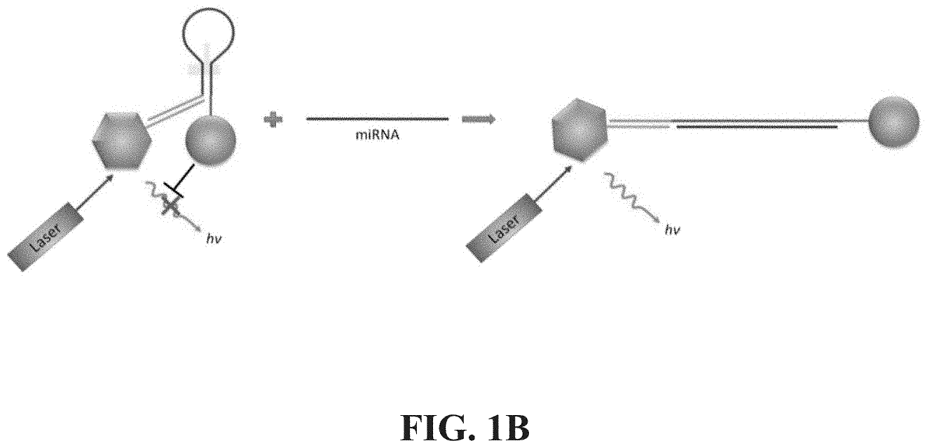

Low background MBs of the present example were built using UCNPs as the reporter (emitting) moiety (in substitution of a fluorophore) and AuNPs as the quencher moiety. In one scheme, represented in FIGS. 1A-1B, a microRNA, such as microRNA 21 (miR-21), was used as a target sequence and was detected to nM level, but it will be understood that the experiment could be carried out with any suitable miRNA sequence, such as those described elsewhere herein. Two types of AuNPs with apophyses (forming non-spherical particles) were synthesized for use as a quencher. One had an absorbance of 540 nm, and one with broad absorption, respectively. The UCNPs were coated with a coupler (a nonylphenol ethoxylate, CO-520), and the coupler-coated UCNPs were linked with single strand DNA to transfer the UCNPs from hydrophobic to hydrophilic. The average ratio between the DNA and the UCNP was about 1:1. In addition, this MB is sensitive and has a low LOD (limit of detection) within a complex detection background.

Nonspherical AuNPs have a different absorption compared with spherical particles. Typical nonspherical AuNPs are larger than 50 nm in diameter, especially those with broad absorption. However, the presently used AuNPs were approximately 25 nm in diameter.

Materials

Gold chloride (HAuC14, 99.9%) was purchased from Strem Chemicals (Newburyport, Mass.). 1-octadecene (ODE; 90%), oleic acid (OA; 90%), oleylamine (70%), NaF (98%), RECl₃.6H₂O (RE=Y, Yb, Er, Tm), MnCl₂.4H₂O, IGEPAL® CO-520, Ascorbic acid (98%), Sodium citrate tribasic dehydrate (99%), Sodium ascorbic (99%), N,N-dimethylformamide (≥99.8%), Polyvinylpyrrolidone (PVP, MW 10,000), and Tris(2-carboxyethyl)phosphine hydrochloride (TCEP, ≥98%) were purchased from Sigma-Aldrich (St. Louis, Mo.). EtOH, hexane (≥99%), and cyclohexane (≥99%) were purchased from Fisher Scientific (Hampton, N.H.). DNA and RNA oligos were purchased from Sigma-Aldrich. DNA and RNA sequences utilized in this Example are shown in Table 1. In Example 1, miR-21 (SEQ ID NO:3) was used as the target analyte. The DNA sequence differs depending on the target sequence.

TABLE 1
Nucleic Acid Sequences used for miRNA 21 MBs

| | |
|---|---|
| DNA on UCNPs | TTTTTTTTTTTTTTTTTTTTT |
| | (SEQ ID NO: 1) |
| DNA on AuNPs | AAAAAAAAAAAAAGTTGTATCGAATAG |
| recognizing miR-21 | TCTGACTACAACTGGG-Thiol |
| | (SEQ ID NO: 2) |
| miR-21 | UAGCUUAUCAGACUGAUGUUGA |
| | (SEQ ID NO: 3) |
| DNA on AuNPs | AAAAAAAAAAAAACGGTTATCGTCGTG |
| recognizing miR- | TCTTTATAACCGGGG-Thiol |
| 195 | (SEQ ID NO: 4) |
| miR-195 | UAGCAGCACAGAAAUAUUGGC |
| | (SEQ ID NO: 5) |

Note:
The DNA probe on AuNPs has three regions: a poly-A region, a target recognizing region, and a 3′ thiol end. The poly-A region complements to the oligo on UCNPs; the hairpin structure of the target recognizing region can disform and complements to the target RNA; the 3′ thiol end has several extra NTs between the target recognizing region and the thiol group, to protect the target recognizing DNA from potential damage in the conjugation process of DNA and AuNPs.

Methods

Synthesis of AuNP Seeds

600 μl of 0.06 M HAuC14 was mixed with 45 mL of H₂O under heating and stirring.

After the temperature reached 95° C., 7 ml of 40 mM sodium citrate was added. About 10 minutes later, the colour of the solution changed from light yellow to light red, and then became dark red. The AuNP seeds were suspended in the solution.

Synthesis of AuNPs

To synthesize AuNPs with 540 nm absorption, 200 μl of 5×AuNPs in EtOH with 1% PVP was stirred overnight. The 5×AuNP solution was mixed with 4 ml of dimethylformamide with 10% PVP and 40 μl of 60 mM HAuC14 under vigorous stirring. 20 μl of 78.8 mM sodium ascorbic was added twice with a gap of 15 minutes. About one (1) hour later, the AuNPs were suspended in the solution.

To synthesize broad absorption AuNPs, 100 μl of 5×AuNPs in EtOH with 1% PVP was stirred overnight. The 5× AuNPs solution was mixed with 4 ml of dimethylformamide with 10% PVP and 40 μl of 60 mM HAuC14 under vigorous stirring. 20 μl of 78.8 mM sodium ascorbic was added twice with a gap of 15 minutes. About one (1) hour later, the AuNPs were suspended in the solution.

DNA Coating of AuNPs

The AuNPs were washed with EtOH for three times and with water twice. The AuNPs were resuspended in 4.5 ml of H₂O. 34 μl of 100 μM DNA on AuNPs and 1 μl of 10 mM TCEP was mixed and incubated at room temperature for one (1) hour. The AuNPs in 4.5 ml of H₂O and the incubated DNA solution was mixed under stirring overnight. The redundant DNA was removed by centrifugation. The DNA-coated AuNPs were resuspended in 4.5 ml of H₂O.

Synthesis of UCNPs

To synthesize UCNPs with a green emission, 2 mmol of YCl₃.6H₂O, 0.45 mmol of YbCl₃.6H₂O, and 0.05 mmol of ErCl₃.6H₂O were dissolved in 13.75 ml OA and 11.25 ml ODE under stirring and vacuuming at 110° C. 10 mmol of NaF was dissolved in 13.75 ml OA and 11.25 ml ODE under stirring and vacuuming 110° C. After dissolving, the above two solutions were mixed and vacuumed for 30 minutes under stirring at 110° C. The reaction system was flushed with N₂ for 20 minutes and was heated in an oil bath (300° C.) under the protection of N₂ for 30 minutes. When the solution cooled down, EtOH with the same volume was added to it and was centrifuged for 20 minutes at 3000 RPM. Later, 10 ml hexane was mixed with the sediment and then kept stand for 10 minutes. After repeating adding EtOH and centrifuging, the UCNPs were kept in 4 ml chloroform.

To synthesize UCNPs with a red emission, 1.6 mmol of MnCl₂.4H₂O, 0.36 mmol of YbCl₃.6H₂O, and 0.04 mmol of ErCl₃.6H₂O were dissolved in 5 ml oleylamine and 20 ml ODE under stirring and vacuuming at 110° C. 6 mmol of KF was dissolved in 5 mL OA and 20 ml ODE under stirring and vacuuming 110° C. After dissolving, the above two solutions were mixed and vacuumed for 30 minutes under stirring at 110° C. The reaction system was flushed with N₂ for 20 minutes and was heated in an oil bath (300° C.) under the protection of N₂ for 120 minutes. When the solution cooled down, EtOH with the same volume was added to it and was centrifuged for 20 minutes at 3000 RPM. Later, 10 ml hexane was mixed with the sediment and then kept stand for 10 minutes. After repeating adding EtOH and centrifuging, the UCNPs were kept in 4 ml chloroform.

To synthesize UCNPs with a blue emission, 1.8 mmol of YCl₃.6H₂O, 0.6 mmol of YbCl₃.6H₂O, and 0.007 mmol of TmCl₃.6H₂O were dissolved in 7 ml OA and 18 ml ODE under stirring and vacuuming at 110° C. 6 mmol of NaF was dissolved in 7 ml OA and 18 ml ODE under stirring and vacuuming 110° C. After dissolving, the above two solutions were mixed and vacuumed for 30 minutes under stirring at 110° C. The reaction system was flushed with N₂ for 20 minutes and was heated in an oil bath (300° C.) under the protection of N₂ for 120 minutes. When the solution cooled down, EtOH with the same volume was added to it and was centrifuged for 20 minutes at 3000 RPM. Later, 10 ml hexane was mixed with the sediment and then kept stand for 10 minutes. After repeating adding EtOH and centrifuging, the UCNPs were kept in 4 ml chloroform.

An alternate method to synthesize UCNPs with blue emission is: 0.75 mmol of YCl₃.6H₂O, 0.25 mmol of YbCl₃.6H₂O, and 0.003 mmol of TmCl₃.6H₂O were dissolved in 3 ml OA and 7.5 ml ODE under stirring and vacuuming at 110° C. 2.5 mmol of NaF was dissolved in 3 ml OA and 7.5 ml ODE under stirring and vacuuming 110° C. After dissolving, the above two solutions were mixed and vacuumed for 30 minutes under stirring at 110° C. The reaction system was flushed with N₂ for 20 minutes and was heated in an oil bath (300° C.) under the protection of N₂ for 90 minutes. When the solution cooled down, EtOH with the same volume was added to it and was centrifuged for 20 minutes at 3000 RPM. Later, 10 ml hexane was mixed with the sediment and then kept stand for 10 minutes. After repeating adding EtOH and centrifuging, the UCNP seeds were kept in 6 ml cyclohexane.

In an alternate method to synthesize UCNPs with blue emission: 0.80 mmol of YCl₃.6H₂O, 0.20 mmol of YbCl₃.6H₂O, and 0.02 mmol of ErCl₃.6H₂O were dissolved in 3 ml OA and 7.5 ml ODE under stirring and vacuuming at 110° C. 2.5 mmol of NaF was dissolved in 3 ml OA and 7.5 ml ODE under stirring and vacuuming 110° C. After dissolving, the above two solutions were mixed with the UCNP seeds in cyclohexane and vacuumed for 30 minutes under stirring at 110° C. The reaction system was flushed with N₂ for 20 minutes and was heated in an oil bath (300° C.) under the protection of N₂ for 90 minutes. When the solution cooled down, EtOH with the same volume was added to it and was centrifuged for 20 minutes at 3000 RPM. Later, 10 ml hexane was mixed with the sediment and then kept stand for 10 minutes. After repeating adding EtOH and centrifuging, the blue UCNPs were kept in 6 ml cyclohexane. UCNPs comprising other compositions may be used in the embodiments of the present disclosure.

DNA Coating of UCNPs

10 ml of cyclohexane with 5 mg of UCNPs was mixed with 500 μl of CO-520 under sonication for 15 minutes. CO-520 can be replaced by other amphiphilic polymers such as Triton™ X-100 and TWEEN® 20, or other suitable surfactants disclosed, for example, in U.S. Pat. No. 9,410,956. The mixture was mixed with 40 μl of 1 nM DNA on UCNPs under vigorous stirring overnight. 10 ml of EtOH was added to wash the DNA-coated UCNPs. The DNA-coated UCNPs were further washed with EtOH twice and with water once. The DNA-coated UCNPs were resuspended in 1 mL of H₂O.

Synthesis of NPMB

1 ml of DNA-coated AuNPs and 200 μl of DNA-coated UCNPs was mixed under stirring for 1 hour. The NPMBs were resuspended in 1 ml of H₂O.

Detection of the Target DNA and RNA

The NPMBs were centrifuged and resuspended in 1 ml of H₂O or 1 ml of spiked plasma, respectively. Spiked plasma is the commercial plasma removed endogenous target RNAs by magnetic beads. Gradient concentration of 1 μl of target DNA or miR-21 was mixed with 199 μl of NPMBs under stirring for one (1) hour. The fluorescence was analyzed by a spectrometer. A 980 nm laser was used as the excitation light, and the excitation of 400 nm to 600 nm was detected.

Results

DNA-AuNPs and DNA-UCNPs were synthesized. The binding efficiency of DNA-AuNPs or DNA-UCNPs was determined by using Alexa Fluor 488 labeled DNAs and quantifying the fluorescent spectra. The binding of DNA does not influence the spectra of the nanoparticles; the absorption light of DNA-AuNPs was 540 nm, and the excitation and emission light of DNA-UCNPs were 980 nm and 540 nm, respectively. The NPMBs were prepared, and the emission spectrum of UCNPs at 540 nm was significantly quenched in the NPMBs and could be recovered by adding target RNAs from 2 μM to 100 nM.

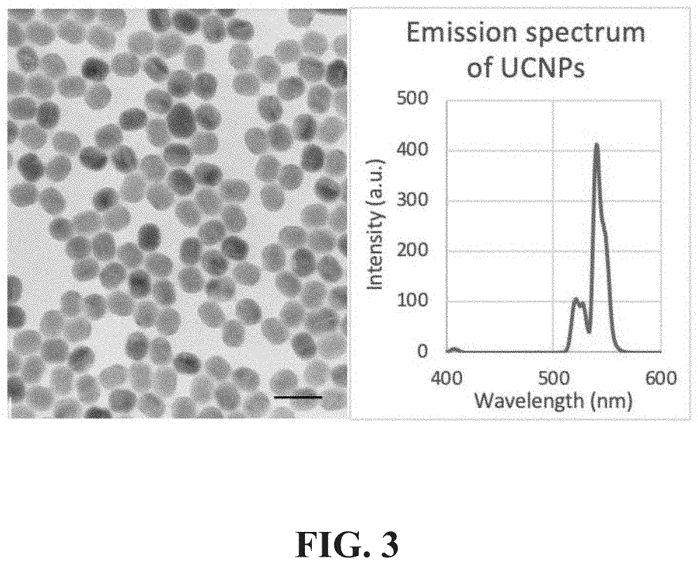

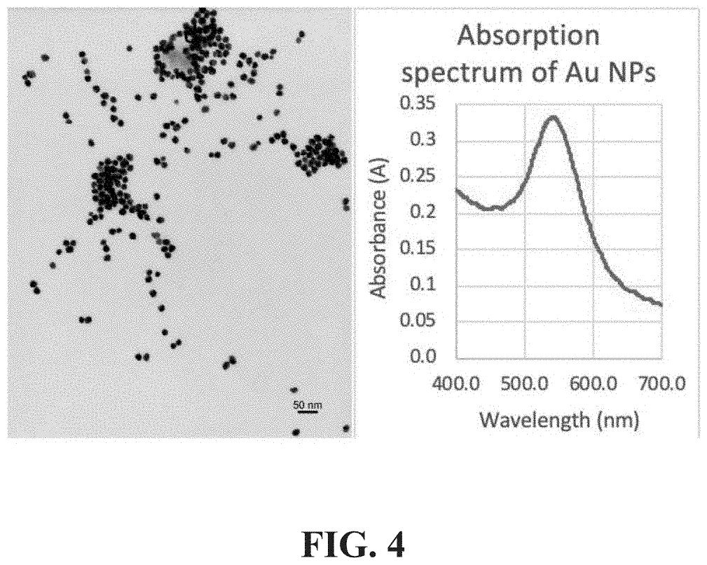

FIG. 3 shows a TEM image (left) and emission spectrum (right) of UCNPs. FIG. 4 shows a TEM image (left) and emission spectrum (right) of AuNPs. The average diameter of UCNPs is 30 nm. The average diameter of the AuNPs is 20 nm. The excitation wavelengths of UCNPs, is 980 nm and the emission peak is at 541 nm. The absorption peak of AuNP is at 546 nm.

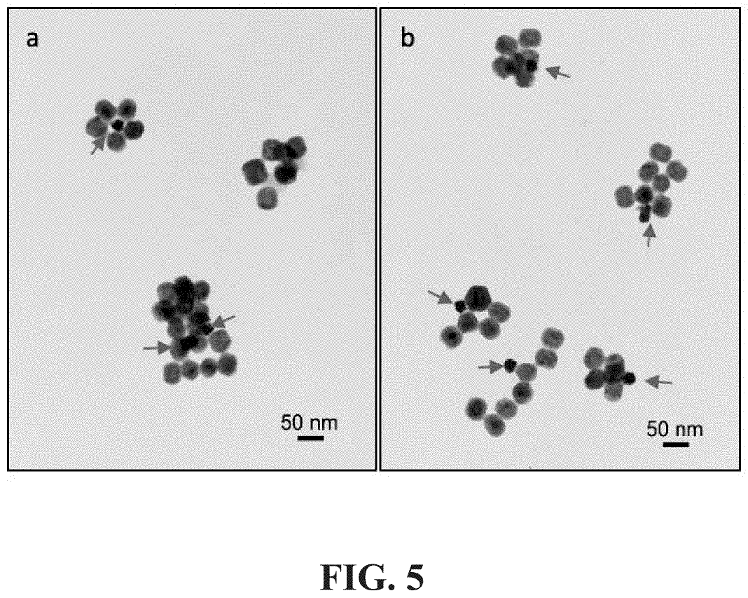

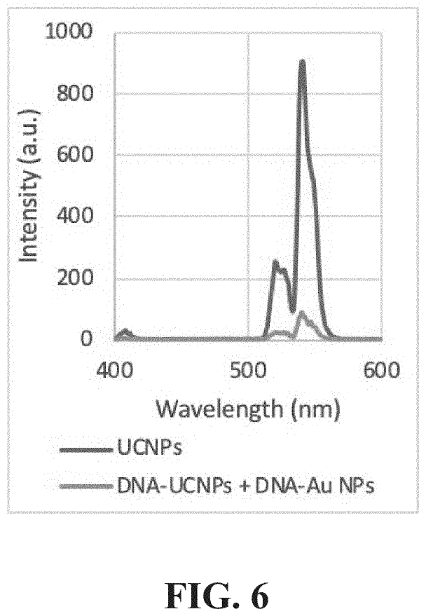

FIG. 5 shows TEM images of NP-based MBs. The arrows indicate the AuNPs. FIG. 6 shows emission spectra of UCNPs (Blue) and UCNP- and AuNP-based MBs (Orange). Same concentrations of UCNPs were detected for spectrum in UCNP solution and NP-based MB solutions. The fluorescence of the MBs was significantly lower than the UCNPs, and the peak does not shift.

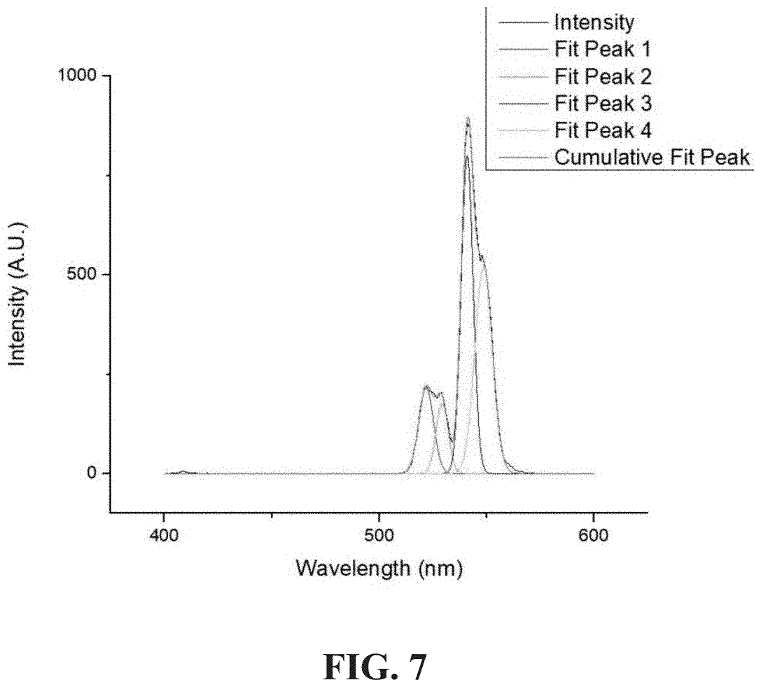

FIG. 7 shows a Peak fit image of UCNP emission spectrum. The emission spectrum of UCNP is fitted as 4 peaks by Origin® 8.6. The cumulative peak area of fit peak 3 and fit peak 4 is used as the main peak area for data analysis. The cumulative peak area of all four fit peaks is used as the peak area.

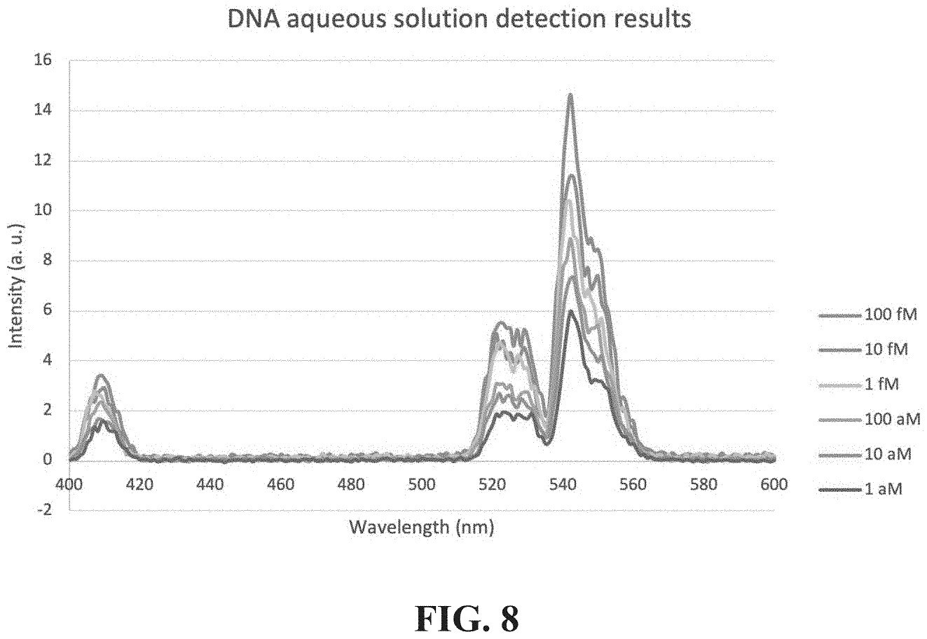

FIG. 8 shows the emission intensity of UCNPs versus target DNA concentration in aqueous solutions. Results indicate that when the target DNA concentrations range from 1 aM to 100 fM, the fluorescence intensity area has a significant logarithmic correlation with the target DNA concentration.

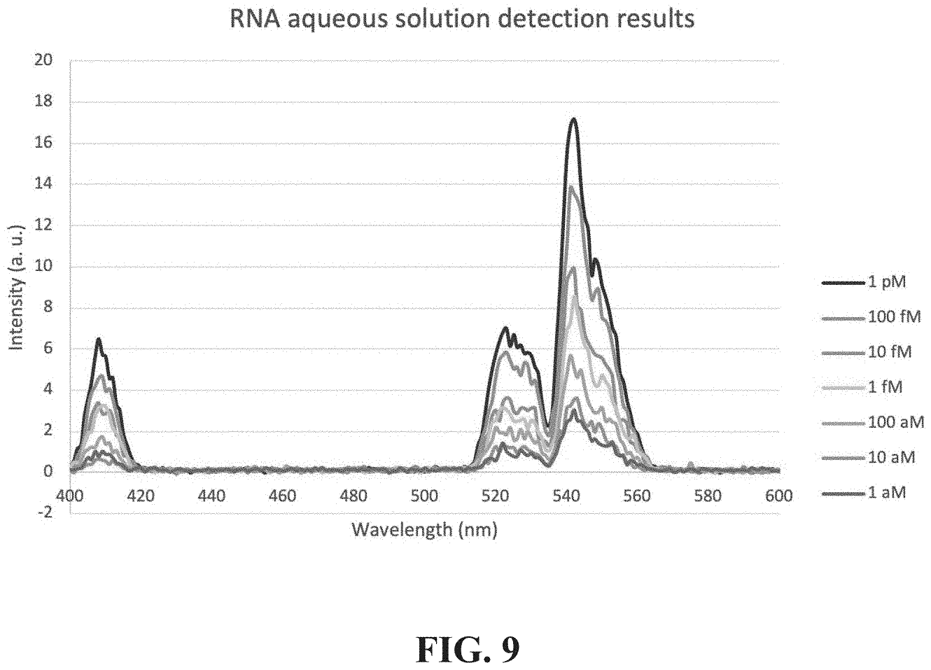

FIG. 9 shows emission intensity of UCNPs versus target RNA concentration in aqueous solution. Results indicate that when the target RNA concentrations range from 1 aM to 1 pM, the fluorescence intensity area has a logarithmic correlation with the target RNA concentration. The correlation is more significant when the target RNA concentrations range from 100 aM to 1 pM. The different detection ranges between DNA and RNA might be caused by RNA degradation.

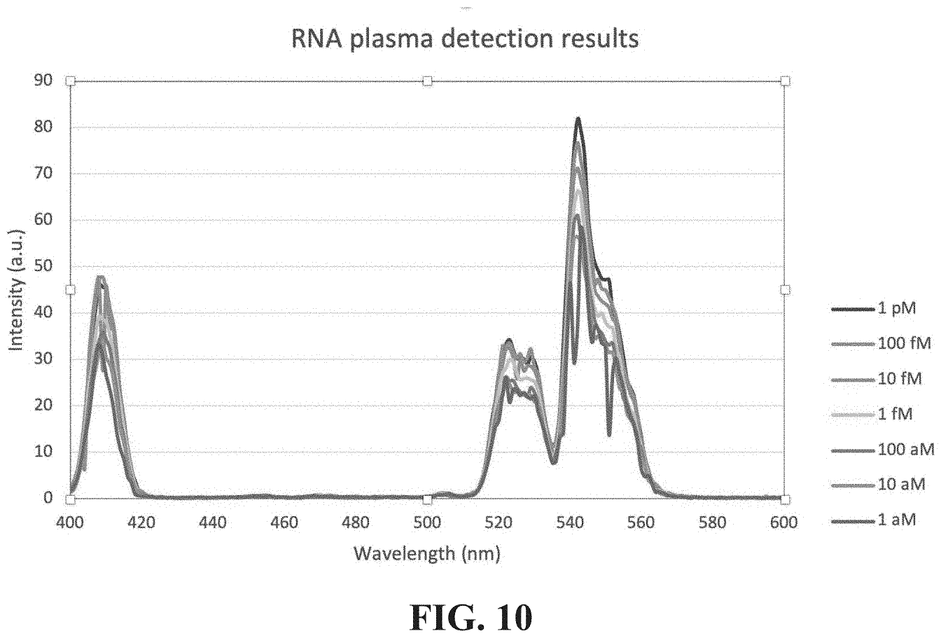

FIG. 10 shows emission intensity of UCNPs versus target RNA concentration in plasma solution. Results indicate that no matter the target RNA concentrations range from 1 aM to 1 pM, the fluorescence intensity area has a logarithmic correlation with the target RNA concentration.

### Example 2

In certain non-limiting embodiments, by using multiple types of UCNPs with different emission wavelengths, the present technology can achieve multiple-target detection at one time, e.g., for use in disease diagnosis. For example, UCNPs (540 nm) correspond to miR-21; blue UCNPs (480 nm) correspond to miR-145; red UCNPs (650 nm) correspond to miR-195. Comparing to the regular range of those miRNAs among healthy people, an increased concentration of miR-21 and a decreased concentration of miR-145 and 195 altogether potentially indicates that the subject tested has developed breast cancer.

In certain embodiments, different types of UCNPs can be utilized within one detection system or complex to achieve multiple-target detection at one time. For example, AuNPs with broad absorption can be modified with an miR-21 recognizing hairpin DNA, then assembled with UCNPs emitting green light to make NPMB-21; another portion of AuNPs with broad absorption can be modified with an miR-195 recognizing hairpin DNA, then assembled with UCNPs emitting red light to make NPMB-195. After mixing the NPMB-21 with NPMB-195 and adding the analyte, the intensity of 540 nm presents the concentration of miR-21, while the intensity of 650 nm presents the concentration of miR-195. In the same way, a third target RNA can be detected by UCNPs emitting blue light and so on.

UCNPs with blue emission can be modified with poly-T oligos by using the same method for the UCNPs with green emission. The UCNPs with green emission and broad absorption AuNPs recognizing miR-21 can be mixed to make NPMBs recognizing miR-21 as described above. Similarly, the UCNPs with blue emission and broad absorption AuNPs recognizing miR-195 can be mixed to make NPMB recognizing miR-195. The two types of NPMBs can be mixed with a gradient concentration of miR-21 and miR-195, and the fluorescence of green and blue light scanned under 980 nm.

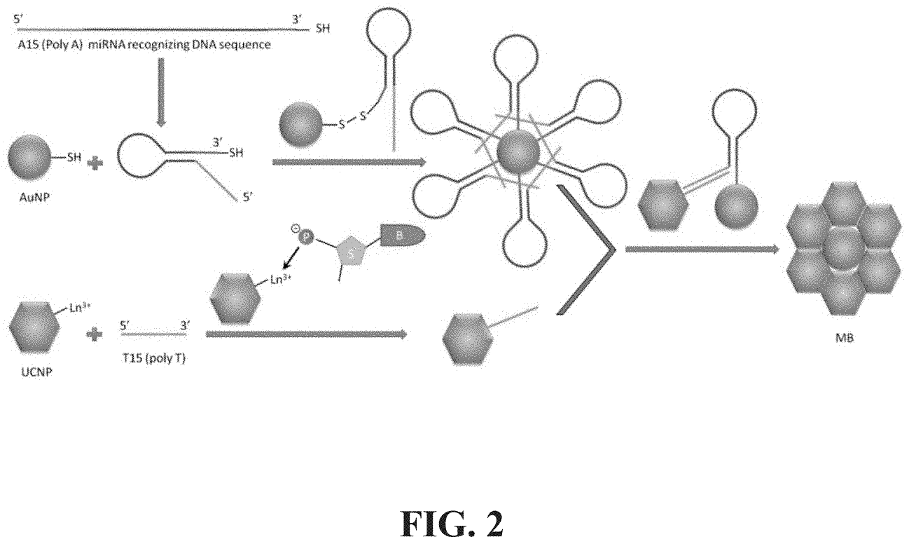

FIG. 2 shows a flow diagram for multiple-UCNP MB formation. The thiol modified hairpin DNA probe has three regions: a poly-A region, a target recognizing region, and a 3′ thiol end. The poly-A region complements to the oligo on UCNPs; the hairpin structure of the target recognizing region can disform and complements to the target RNA; the 3′ thiol end has several extra nucleotides between the target recognizing region and the thiol group, to protect the target recognizing DNA from potential damage in the conjugation process of DNA and AuNPs. The thiol modified hairpin DNA probe conjugates with AuNP through a thiol group. The average number of DNA probes on each AuNP is 75. The UCNPs bind with a poly-T DNA oligo by lanthanide ion and phosphate group interaction. The average number of DNA probes on each UCNP is 1. The DNA-modified AuNP and the DNA-modified UCNP are assembled by the poly-A and the poly-T hybridization. The UCNP is quenched by AuNP without the target RNA; when the target contacts the MB, the hairpin DNA probe binds complementarily to the target RNA, and the UCNP fluorescence will be detected.

Table 2 shows a non-exhaustive list of exemplary RNA molecules that can be identified using the presently disclosed methods. As noted above, RNA sequences which may be identified include, but are not limited to, the micro RNAs shown in Table 1 of U.S. Pat. No. 10,093,967.

TABLE 2
Small Non-Coding RNA Cancer Biomarkers

| | | | | Cancer |
|---|---|---|---|---|
| RNA | RNA type | Regulation | Sample | type |
| miR-21 | miRNA | Up-regulating | Serum | Breast |
| | | | | cancer |
| miR-7 | miRNA | Down-regulating | Cell line; | Breast |
| | | | tumor | cancer |
| miR-34a | miRNA | Down-regulating | Cell line; | Breast |
| | | | tumor | cancer |
| miR-92a | miRNA | Down-regulating | Serum | Breast |
| | | | | cancer |
| miR-141 | miRNA | Down-regulating | Animal model | Breast |
| | | | Blood | cancer |
| miR-200 | miRNA | Down-regulating | Cell line; | Breast |
| | | | animal model | cancer |
| | | | Blood | |
| U50 | snoRNA | Down-regulating | Cell line | Breast |
| | | | | cancer |
| piR-651 | piRNA | Up-regulating | Cell line | Breast |
| | | | | cancer |
| piR-36026 | piRNA | Up-regulating | Cell line; | Breast |
| | | | animal model | cancer |
| miRNA-141 | miRNA | Up-regulating | Serum | Prostate |
| | | | | cancer |
| miR1245 | miRNA | Up-regulating | Cell line; | Prostate |
| | | | tumor | cancer |
| miR-1 | miRNA | Down-regulating | Cell line; | Prostate |
| | | | tumor | cancer |
| miR-23b | miRNA | Down-regulating | Cell line; | Prostate |
| | | | tumor | cancer |
| miR-24-1 | miRNA | Down-regulating | Cell line; | Prostate |
| | | | tumor | cancer |
| miR-27b | miRNA | Down-regulating | Cell line; | Prostate |
| | | | tumor | cancer |
| U50 | snoRNA | 2 bps deletion | Cell line; | Prostate |
| | | | tumor; blood | cancer |
| miR-9 | miRNA | Up-regulating | Tumor | Lung |
| | | | | cancer |
| miR-21 | miRNA | Up-regulating | Cell line; | Lung |
| | | | tumor | cancer |
| miR-126 | miRNA | Up-regulating | Tumor | Lung |
| | | | | cancer |
| miR-141 | miRNA | Up-regulating | Cell line; | Lung |
| | | | tumor | cancer |
| miR-150 | miRNA | Up-regulating | Tumor | Lung |
| | | | Plasma | cancer |
| miR-200 | miRNA | Up-regulating | Cell line; | Lung |
| | | | tumor | cancer |
| miR-486 | miRNA | Up-regulating | Plasma | Lung |
| | | | | cancer |
| miR-1290 | miRNA | Up-regulating | Tumor | Lung |
| | | | serum | cancer |
| miR-152 | miRNA | Down-regulating | Plasma | Lung |
| | | | | cancer |
| miR-375 | miRNA | Down-regulating | Tumor; | Lung |
| | | | plasma | cancer |
| miR-638 | miRNA | Down-regulating | Tumor | Lung |
| | | | serum | cancer |
| let-7c | miRNA | Down-regulating | Plasma | Lung |
| | | | | cancer |
| SNORD33 | snoRNA | Up-regulating | Plasma | Lung |
| | | | | cancer |
| SNORD36 | snoRNA | Up-regulating | Plasma | Lung |
| | | | | cancer |
| SNORD76 | snoRNA | Up-regulating | Plasma | Lung |
| | | | | cancer |
| miR-186-5p | miRNA | Up-regulating | Cell line; | colon |
| | | | tumor | cancer |
| miR-362 | miRNA | Up-regulating | Cell line; | stomach |
| | | | tumor | cancer |
| miR-372 | miRNA | Up-regulating | Cell line | stomach |
| | | | | cancer |
| miR-10a | miRNA | Down-regulating | Cell line | stomach |
| | | | | cancer |
| miR-133b | miRNA | Down-regulating | Cell line; | stomach |
| | | | tumor | cancer |
| miR-135a | miRNA | Down-regulating | Cell line; | stomach |
| | | | tumor | cancer |
| miR-337-3p | miRNA | Down-regulating | Cell line: | stomach |
| | | | tumor | cancer |
| piR-651 | piRNA | Up-regulating | Cell line | stomach |
| | | | | cancer |
| pi-823 | piRNA | Down-regulating | Cell line | stomach |
| | | | | cancer |
| PIWIL3 | piRNA | Down-regulating | Cell line; | ovarian |
| | | | tumor | cancer |

Note:
long micro RNA (miRNA),
small nucleolar RNA (snoRNA),
Piwi-interacting RNA (piRNA)

As shown above, a novel strategy for detecting small DNA and RNA molecules was developed. In this strategy, MBs were first built up with inorganic nanoparticles as both fluorophore and quencher. Another advantage of this strategy is that it provides less background noise compared to the other MB-based nucleic acid detection systems.

Therefore, in at least certain non-limiting embodiments, the present disclosure is directed to an oligonucleotide probe comprising a nucleic acid probe sequence comprising a hairpin loop; a reporter moiety linked to a first end of the nucleic acid probe sequence, wherein the reporter moiety is an upconversion nanoparticle; and a quencher moiety linked to a second end of the nucleic acid probe sequence; wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence. The nucleic acid probe sequence may optionally be a DNA sequence. The complementary target nucleic acid sequence may be a DNA sequence or an RNA sequence. The complementary target nucleic acid may optionally be a micro RNA. The gold nanoparticle may optionally comprise apophyses.

In another non-limiting embodiment, the present disclosure is directed to a method of screening a sample for a target nucleic acid sequence, comprising the steps of obtaining an oligonucleotide probe comprising (1) a nucleic acid probe sequence comprising a hairpin loop; (2) a reporter moiety linked to a first end of the nucleic acid probe sequence, wherein the reporter moiety is an upconversion nanoparticle; and (3) a quencher moiety linked to a second end of the nucleic acid probe sequence, wherein the quencher moiety is a gold nanoparticle, wherein the nucleic acid probe sequence is complementary to the target nucleic acid sequence, and wherein the reporter moiety is quenched by the quencher moiety; combining the oligonucleotide probe with the sample, thereby forming an oligonucleotide probe-target nucleic acid complex when the target nucleic acid sequence is present in the sample, whereby the reporter moiety becomes unquenched when the nucleic acid probe sequence binds to the target nucleic acid sequence; and analyzing the sample to detect or measure the oligonucleotide probe-target nucleic acid sequence complex. The sample may optionally be of biological origin. The nucleic acid probe sequence may optionally comprise a DNA sequence. The target nucleic acid sequence may be a DNA sequence or an RNA sequence. The target nucleic acid sequence may optionally be a micro RNA sequence. The gold nanoparticle may optionally comprise apophyses.

In another non-limiting embodiment, the present disclosure is directed to a kit, comprising a first oligonucleotide probe, comprising a nucleic acid probe sequence having a hairpin loop; a reporter moiety linked to a first end of the nucleic acid probe sequence, wherein the reporter moiety is an upconversion nanoparticle; and a quencher moiety linked to a second end of the nucleic acid probe sequence, wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence. The kit further comprises a second oligonucleotide probe, comprising a nucleic acid probe sequence comprising a hairpin loop, wherein the nucleic acid probe sequence of the second oligonucleotide probe is different from the nucleic acid probe sequence of the first oligonucleotide probe; a reporter moiety linked to a first end of the nucleic acid probe sequence, wherein the reporter moiety is an upconversion nanoparticle, and wherein the reporter moiety of the second oligonucleotide probe is different from the reporter moiety of the first oligonucleotide probe; and a quencher moiety linked to a second end of the nucleic acid probe sequence, wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence. The nucleic acid probe sequence of at least one of the first and second oligonucleotide probes optionally comprises a DNA sequence. The complementary target nucleic acid sequence of at least one of the first and second oligonucleotide probes may optionally be an RNA sequence, wherein the RNA optionally is a micro RNA. The complementary target nucleic acid sequence of at least one of the first and second oligonucleotide probes may optionally be a DNA sequence. The gold nanoparticle of at least one of the first and second oligonucleotide probes may optionally comprise apophyses.

In another non-limiting embodiment, the present disclosure is directed to a method of screening a sample for at least two target nucleic acid sequences, comprising combining the first and second oligonucleotide probes of the kit with the sample, thereby forming a first oligonucleotide probe-target nucleic acid complex when the first target nucleic acid sequence is present in the sample, whereby the reporter moiety of the first oligonucleotide probe becomes unquenched when the nucleic acid probe sequence binds to the first target nucleic acid sequence, and thereby also forming a second oligonucleotide probe-target nucleic acid complex when the second target nucleic acid sequence is present in the sample, whereby the reporter moiety of the second oligonucleotide probe becomes unquenched when the nucleic acid probe sequence binds to the second target nucleic acid sequence. The method further comprises the step of analyzing the sample to detect or measure the first oligonucleotide probe-target nucleic acid sequence complex and the second oligonucleotide probe-target nucleic acid sequence complex.

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the components, formulation of the various compositions described herein, the methods described herein, or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. An oligonucleotide probe, comprising:
a nucleic acid probe sequence comprising a hairpin loop;
a reporter moiety linked to a first end of the nucleic acid probe sequence, wherein the reporter moiety is an upconversion nanoparticle; and
a quencher moiety linked to a second end of the nucleic acid probe sequence, wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence.

2. The oligonucleotide probe of claim 1, wherein the nucleic acid probe sequence comprises a DNA sequence.

3. The oligonucleotide probe of claim 1, wherein the complementary target nucleic acid sequence is an RNA sequence.

4. The oligonucleotide probe of claim 3, wherein the RNA is a micro RNA.

5. The oligonucleotide probe of claim 1, wherein the complementary target nucleic acid sequence is a DNA sequence.

6. The oligonucleotide probe of claim 1, wherein the gold nanoparticle comprises apophyses.

7. A method of screening a sample for a target nucleic acid sequence, the method comprising the steps of:
obtaining an oligonucleotide probe comprising (1) a nucleic acid probe sequence comprising a hairpin loop; (2) a reporter moiety linked to a first end of the nucleic acid probe sequence, wherein the reporter moiety is an upconversion nanoparticle; and (3) a quencher moiety linked to a second end of the nucleic acid probe sequence, wherein the quencher moiety is a gold nanoparticle, wherein the nucleic acid probe sequence is complementary to the target nucleic acid sequence, and wherein the reporter moiety is quenched by the quencher moiety;
combining the oligonucleotide probe with the sample, thereby forming an oligonucleotide probe-target nucleic acid complex when the target nucleic acid sequence is present in the sample, whereby the reporter moiety becomes unquenched when the nucleic acid probe sequence binds to the target nucleic acid sequence; and
analyzing the sample to detect or measure the oligonucleotide probe-target nucleic acid sequence complex.

8. The method of claim 7, wherein the sample is of biological origin.

9. The method of claim 7, wherein the nucleic acid probe sequence comprises a DNA sequence.

10. The method of claim 7, wherein the target nucleic acid sequence is an RNA sequence.

11. The method of claim 10, wherein the RNA is a micro RNA.

12. The method of claim 7, wherein the target nucleic acid sequence is a DNA sequence.

13. The method of claim 7, wherein the gold nanoparticle comprises apophyses.

14. A kit, comprising:
a first oligonucleotide probe, comprising:
a nucleic acid probe sequence comprising a hairpin loop;
a reporter moiety linked to a first end of the nucleic acid probe sequence, wherein the reporter moiety is an upconversion nanoparticle; and
a quencher moiety linked to a second end of the nucleic acid probe sequence, wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence; and
a second oligonucleotide probe, comprising:
a nucleic acid probe sequence comprising a hairpin loop, wherein the nucleic acid probe sequence of the second oligonucleotide probe is different from the nucleic acid probe sequence of the first oligonucleotide probe;
a reporter moiety linked to a first end of the nucleic acid probe sequence, wherein the reporter moiety is an upconversion nanoparticle, and wherein the reporter moiety of the second oligonucleotide probe is different from the reporter moiety of the first oligonucleotide probe; and
a quencher moiety linked to a second end of the nucleic acid probe sequence, wherein the quencher moiety is a gold nanoparticle, wherein the quencher moiety quenches the reporter moiety when the nucleic acid probe sequence is not bound to a complementary target nucleic acid sequence, and wherein the reporter moiety becomes unquenched when the nucleic acid probe sequence binds to a complementary target nucleic acid sequence.

15. The kit of claim 14, wherein the nucleic acid probe sequence of at least one of the first and second oligonucleotide probes comprises a DNA sequence.

16. The kit of claim 14, wherein the complementary target nucleic acid sequence of at least one of the first and second oligonucleotide probes is an RNA sequence.

17. The kit of claim 16, wherein the RNA is a micro RNA.

18. The kit of claim 14, wherein the complementary target nucleic acid sequence of at least one of the first and second oligonucleotide probes is a DNA sequence.

19. The kit of claim 14, wherein the gold nanoparticle of at least one of the first and second oligonucleotide probes comprises apophyses.


SEQUENCE LISTING (5 sequences)

SEQ ID NO: 1 - DNA, 21 bp, Artificial sequence
  oligo probe - DNA on UCNPs
ttttttttttttttttttttt

SEQ ID NO: 2 - DNA, 43 bp, artificial sequence
  oligo probe - DNA on AuNPs recognizing miR-21
  misc_feature (43)..(43): thiol
aaaaaaaaaaaaagttgtatcgaatagtctgactacaactggg

SEQ ID NO: 3 - RNA, 22 nt, Homo sapiens
uagcuuaucagacugauguuga

SEQ ID NO: 4 - DNA, 42 bp, artificial sequence
  oligo probe - DNA on AuNPs recognizing miR-195
  misc_feature (42)..(42): Thiol
aaaaaaaaaaaaacggttatcgtcgtgtctttataaccgggg

SEQ ID NO: 5 - RNA, 21 nt, Homo sapiens
uagcagcacagaaauauuggc